(19) United States
(12) Patent Application Publication
(10) Pub. No.: US 2006/0084923 A1
(43) Pub. Date: Apr. 20, 2006

(54) Gastrostomy feeding tube cushion device

(76) Inventor: Karen Lotartaro, Staten Island, NY (US)

Correspondence Address: DARBY & DARBY P.C., P. O. BOX 5257, NEW YORK, NY, 10150-5257, US

(21) Appl. No.: 10/966,724

(22) Filed: Oct. 15, 2004

(51) Int. Cl. A61M 5/32

(52) U.S. Cl. 604/179

(57) ABSTRACT

The invention is directed to a cushion device for a user having a gastrostomy tube or gastrostomy button protruding from an anterior region of the user's torso, and a method of using such a device with a individual to increase comfort and tolerance with regard to the tube and/or button when placed on the anterior torso and a method to encourage the physical development of a child having such a tube and/or button. By way of example, the cushion device includes an elongated body having opposing end sections, the end sections releasably securable to one another when the body is placed around the user, wherein the body includes a padding having a thickness at least as great as the button or the gastrostomy tube, and wherein the padding further includes an open area to accommodate the button or the gastrostomy tube.

### TECHNICAL FIELD

The present invention relates to gastrostomy feeding tubes and, more particularly, to a cushion device and method for a gastrostomy tube and/or button protruding from the anterior region of a user's torso.

### BACKGROUND

Forty to seventy percent of infants born prematurely or infants with chronic medical conditions show evidence of feeding problems. One common way of dealing with these problems is through the use of feeding tubes. One type of feeding tube, a gastrostomy feeding tube (g-tube), is a tube that is surgically placed into the stomach through the abdominal wall. The surgery involves cutting a hole (stoma) in the skin and into the stomach. Then the stomach is gently attached to the abdominal wall and the g-tube is fitted into the stoma. The g-tube is typically held in place by a disc or water filled balloon that has a one-way valve to keep stomach contents in and air, etc., out. Once the gastrostomy site is well healed and the tube has been in place for a minimum of eight weeks, it is possible to change to a skin level feeding device commonly known as a “button”. A button sits directly on the skin and has a connector valve for the attachment of a removable tube used during feeding time. The feedings are in liquid form and there are several ways to feed through the tube. One common way is by bolus, which is a larger amount of feeding given three-six times per day. Sometimes it is tube. After feeding, the tube can be disconnected from the button or stay connected with the end closed off by another valve that looks similar to the valve used to inflate a beach ball. There are several types of buttons, including those that may have a disc, clamp, or crossbar on the outside to keep them in place. The most common are the Button made by Bard and the MIC-key made by Medical Innovations Corporation. Most buttons protrude from the skin approximately ½ inch and are approximately an inch in width.

There are a variety of reasons why children may require a g-tube/button. The primary indication for gastrostomy is the child's inability to take adequate nutrition or liquids by mouth for growth and development. In some cases, tube feedings accompany oral feedings to ensure nutritional needs are being met. A g-tube/button will reduce the stress level associated with trying to provide the nutrition needed for growth and development by providing nourishment directly through the tube to the stomach. Severe feeding and swallowing problems in infants and children are rarely seen as isolated disorders. The majority of the time it is related to neurologic, respiratory, gastrointestinal (digestive), mechanical and/or structural problems. Disorders which may be associated with feeding and swallowing difficulties, include but are not limited to: cerebral palsy, muscular dystrophy, encephalopathies (brain disorders), traumatic brain injuries, neoplasms, “failure to thrive”, congenital or acquired anatomic and structural problems (cleft palate), Down Syndrome, prematurity, genetic disorders, gastrointestinal illness, and congenital cardiac anomalies. Depending on the severity of the disorder and feeding/swallowing difficulties, a child may need tube feedings for short term, long term (several years) or for a lifetime.

Children with g-tubes/buttons usually have difficulty lying on their stomach. The button is usually located on the left side of the body. It typically protrudes approximately ½ inch from the skin. Since the “button” protrudes approximately ½ inch from the body and is located on the left side of the anterior aspect of the trunk, when a child is placed on his/her stomach, the child's body weight provides an uneven pressure to the “button”. The increased pressure at the “button” site commonly causes discomfort and pain. The pressure may even cause the “button” to shift in position, which may increase the risk of dislodging.

This difficulty with stomach placement can cause problems with achieving developmental milestones because the child will avoid lying on his/her stomach. It also interferes with positioning during therapy and playtime. Children need to lie on their stomach to learn how to roll, transition in/out of positions, pivot on their stomach, and crawl to explore their environment. During playtime, lying on their stomach can assist with improving head control and strengthening their trunk and extremities.

Many children who are born prematurely don't have the opportunity to develop flexor patterns of movement (which develops the last couple of months within the womb). These children often present with excessive extensor tone (arching their back and keeping their arms and legs extended) because the flexor tone has not been fully developed. The flexion pattern allows an infant to bring extremities towards the midline of the body and assists with flexion of the trunk and extremities for mobility. Therefore, these children need to develop this pattern of movement outside the womb. There needs to be a balance between the extensor and flexor tone to promote mobility and function. Lying on their stomach is crucial for this to occur. Children with abnormal muscle tone (hypotonic-decreased muscle tone and hypertonic-increased muscle tone) therapeutically would benefit from weight bearing activities to assist with “normalizing” muscle tone. Lying on their stomach promotes weight bearing through extremities when attempting to push up onto arms, pivoting on stomach and crawling.

All of these activities are difficult to do for a child who experiences pain or discomfort when placed on his or her stomach as a result of the protruding g-tube/button. Hence, there is a need for a system which would minimize this discomfort and allow children and others to comfortably interact and play on their stomach despite the presence of a g-tube/button.

### SUMMARY OF THE INVENTION

It is an object of the invention to provide a cushion device for an individual having a g-tube/button.

It is another object of this invention to provide a system and method for relieving pressure and discomfort of the g-tube/button site when an individual is lying on his or her stomach.

It is yet another object of this invention to provide a system and method for encouraging the physical development of a child having a g-tube/button by allowing the child to crawl, roll, pivot and/or play on his or her stomach.

In accordance with a first aspect of the invention, there is provided a cushion device for a gastrostomy tube or gastrostomy button protruding from an anterior section of a user's torso, the cushion comprising:

(a) an elongated body having opposing end sections, the end sections releasably securable to one another when the body is placed around the user, the body including a padding having a thickness at least as great as the button or the gastrostomy tube, the padding further including an open area to accommodate the button or the gastrostomy tube.

In accordance with another aspect of the invention, there is provided a method for encouraging the physical development of a child having a gastrostomy tube or a gastrostomy button protruding from an anterior region of the child's torso, the method comprising the steps of:

(a) providing a cushion device around the child's torso, the cushion device including an elongated body having opposing end sections, the end sections releasably securable to one another when the body is placed around the child, the body including a padding having a thickness at least as great as the button or the gastrostomy tube, the padding further including an open area to accommodate the button or the gastrostomy tube;

(b) securing the cushion device around the child so that the open area is disposed over the button or tube;

(c) placing the child on the child's anterior region; and

(d) improving mobility and muscle strengthening by allowing the child to perform an activity selected from the group consisting of crawling, rolling, playing and pivoting.

In accordance with yet another aspect of the invention, there is provided a method for increasing an individual's comfort with respect to a gastostomy tube or gastostomy button protruding from an anterior region of the individual's torso, the method comprising the steps of:

(a) securing a cushion device around the torso, the cushion device including a padding having a thickness at least as great as the button or the gastrostomy tube, the padding further including an open area to accommodate the button or the gastrostomy tube when placed over the button or tube.

These and other aspects, features and arrangements of the invention can be appreciated from the accompanying Drawings and Detailed Description of an Exemplary Embodiment.

### BRIEF DESCRIPTION OF THE DRAWINGS

FIG. 1 is a top view of a child having a gastrostomy tube/button protruding from the child's anterior torso region;

FIGS. 2a-2c are front views of a cushion device according to a first, second and third embodiment of the present invention;

FIG. 2d is a rear view of a cushion device according to a fourth embodiment of the present invention;

FIG. 3a is an overview of a cushion device according to one embodiment of the present invention as it would be secured around a user;

FIG. 3b is a side view of the cushion device shown in FIG. 3a;

FIG. 4 is a side view of a cushion device according to one aspect of the present invention with an open area disposed over a gastrostomy button protruding from a user's stomach;

FIG. 5a is a front view of a user wearing a cushion device according to one embodiment of the present invention;

FIG. 5b is a rear view of the user shown in FIG. 5a;

FIG. 6 is an overview of a cushion device having adjustable shoulder straps according to one embodiment of the present invention; and

FIG. 7 is an overview of a cushion device having a tube securer according to one embodiment of the present invention.

### DETAILED DESCRIPTION OF A PREFERRED EMBODIMENT

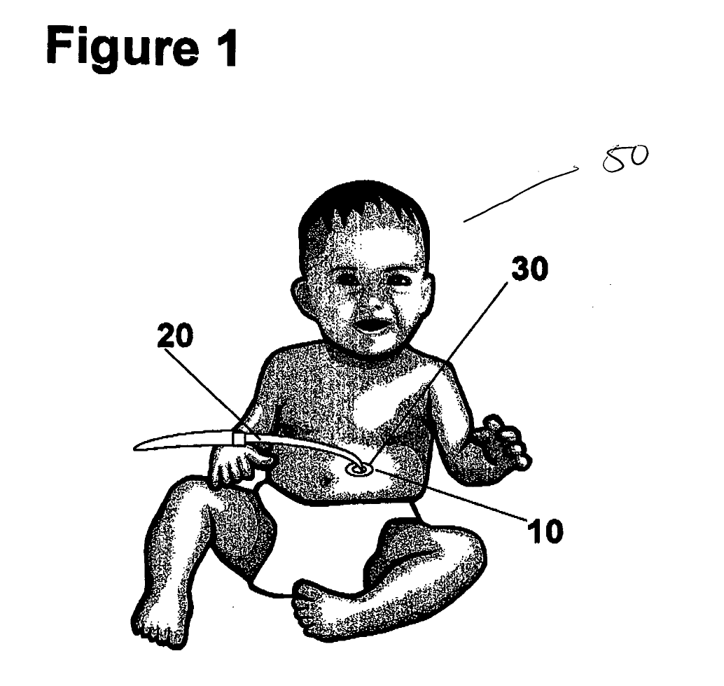

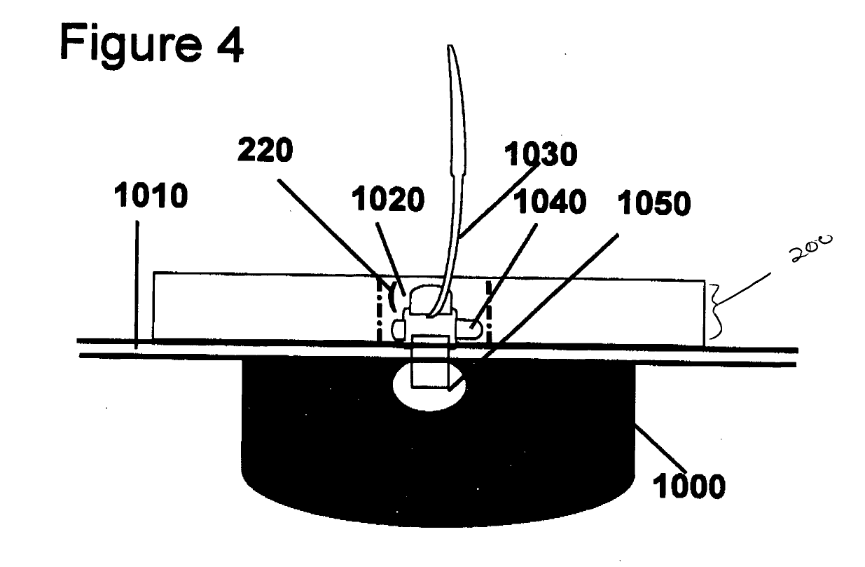

By way of overview and introduction, the present invention provides for a cushion device for a g-tube/button protruding from an anterior region of a user's torso. The present invention is most beneficially used with children, such as a child 50 having a protruding g-tube/button as shown in FIG. 1. Shown on the child's anterior torso is a gastrostomy site 10, at which site may protrude a gastrostomy button 30, such as a PEG style or MIC-key style button, or a gastrostomy tube 20. One type of button, a MIC-key type button 1020 is further illustrated in FIG. 4. This type of button typically includes a detachable feeding tube 1030, a detachable extension 1040 and a balloon 1050 to hold the button in place. As shown, the balloon 1050 is placed below the stomach muscle and abdominal wall 1010 to hold the button/tube 1020 to the individual's stomach 1000.

As is readily apparent, a child having a g-tube/button can not be placed on his or her stomach without experiencing discomfort or pain as a result of the pressure on the g-tube/button. In order to minimize such difficulties, the present invention is directed to a cushion device for the g-tube/button, which cushion device includes an open area to cushion and protect the g-tube/button. The cushion device can be placed and secured around the user's torso and is particularly beneficial when the user is placed on his or her stomach.

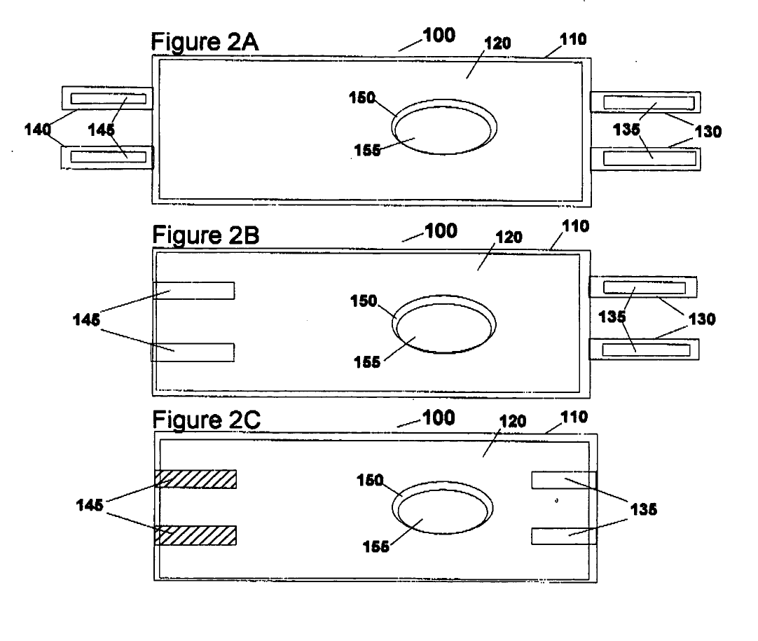

Referring to FIG. 2a, one embodiment for the cushion device 100 is illustrated. The cushion device 100 includes an elongated body portion 110 which is preferably made of felt, although any other material may instead be used. The cushion device 100 includes a padding 120, which padding may be integrally formed with the elongated body 110 such that the padding and body are a single piece; it may also be attached to the body 10 or it may be housed within a pocket (not shown) formed within the body 110. Additionally, the padding 120, the elongated body 110 or both may be placed within a removable cover (not shown), such that the cover can be placed over the padding 120 and body 110 during use, but can be removed for cleaning as necessary. The elongated body 110 includes an open area 150 within the body 110. Similarly, the padding 120 includes an open area 155 generally disposed in the same vicinity as the elongated body open area 150, and, in the circumstance where a cover (not shown) is used, the cover will also include a similarly disposed open area. It will be appreciated that a single open area can be used for both the padding 120 and the body 110. The open area may be formed by cutting a generally circular region in the padding 120 and body 110, or it can be formed by any of a variety of other known methods. The open area 150, 155 may be of any size and shape (oval shape is shown) provided the open area 150, 155 is large enough to accommodate the width of the g-tube/button 1020 protruding from the gastrostomy site 10. The cushion device 100 also has a thickness 200 that is preferably equal to or greater than the height 220 of the g-tube/button that protrudes from the stomach wall 1010.

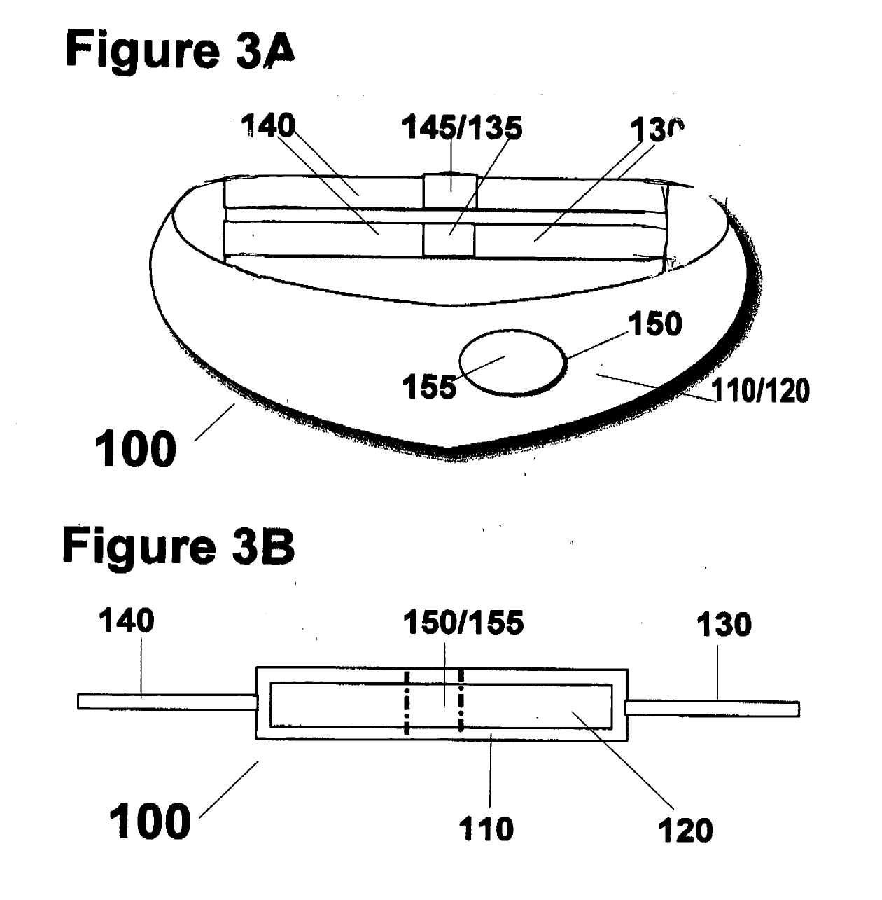

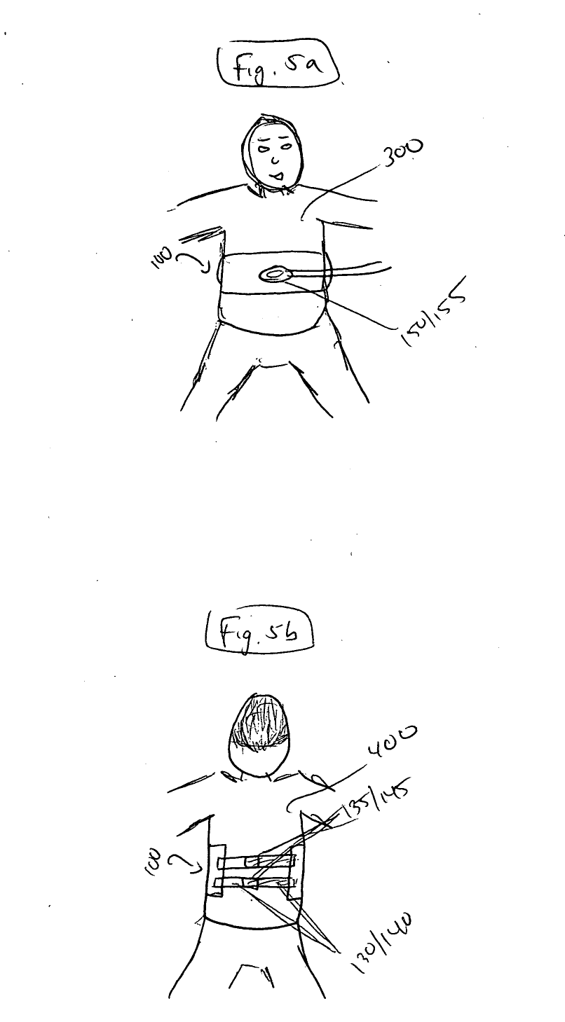

According to one embodiment of the present invention, the cushion device 100 includes one or more straps 130/140 protruding from each of the opposing ends of the elongated body 110. In the example shown in FIG. 2a, two straps 130 are shown protruding from the right side of the body 110 and two straps 140 are shown protruding from the left side of the body 110. Each of the straps 130/140 preferably includes an attachment mechanism such that the straps on one side can be releasably secured to the straps on the other side. In this manner, and as illustrated in FIG. 3a and FIGS. 5a and 5b, the right and left side straps can be secured to one another during use so as to tighten the cushion device 100 around the user, and then disconnected so that the cushion device 100 can be removed as desired. One preferred type of attachment mechanism includes opposing Velcro® strips 135/145 connected to each of the straps 130/140. The strips 135/145 may be connected to the straps 130/140 by any of a variety of known methods. Alternatively, hooks, buttons, snaps, zippers or any other known attachment mechanisms may be used.

According to another embodiment, as illustrated in FIG. 2b, the cushion device 100 includes one or more right side straps 130 with their associated attachment mechanisms 135, but no left side straps. Instead, the left side attachment mechanisms 145 are included directly on the body 110 such that the right side straps 130 are releasably secured to the body 110. Alternatively, left side straps may be used and the right side attachment mechanism may be secured to the body 110. According to yet another embodiment shown in FIG. 2c, both the right and left side straps are eliminated and the right and left side attachment mechanisms 135/145 are included directly on the body 110 such that the right and left sides of the body 110 secure to one another.

According to another embodiment, as illustrated in FIG. 2d, the elongated body 110 of the cushion device 100 includes an added section of adhesion material 152 such that the material 152 may be placed in contact with a wearer's skin when the device is utilized. The added material 152 may lightly adhere to the skin without irritating or pulling on the skin and can assist with maintaining proper positioning of the cushion device 100 on the wearer. Preferably, the added material 152 is made of medical-grade silicone or gel-like fibers, although any type of suitable material may be used.

The operation of the cushion device of the present invention is illustrated in FIGS. 5a and 5b. The user of the present invention has a g-tube/button which protrudes from the anterior region 300 of the user's torso. The cushion device 100 is placed over the anterior region 300 such that the open area 150/155 is disposed over the g-tube/button 20/30. Because the cushion device 100 has a thickness 200 that is at least as great as the thickness 220 of the g-tube/button, the cushion device 100 provides a generally uniform layer of cushioning over the user's anterior region, which cushioning fully covers the g-tube/button 20/30. The cushion device 100 is wrapped around the user's anterior region and the right and left side straps 130/140 are draped around the user's posterior region 400. The straps 130/140 are pulled tight so that the cushion device 100 is securely but comfortably fastened around the user, and then the straps 130/140 are releasably secured to one another using the right and left side attachment mechanisms 135/145. Once the cushion device 100 is placed around the user, the user may be comfortably placed on his or her stomach without experiencing the pain and/or discomfort that would normally be associated with being placed in that position without the use of the cushion device 100.

It will be appreciated that the thickness 200 of the cushion device 100 can be varied to accommodate the different sizes of g-tubes/buttons that are, or will be sold. A thickness of approximately ¾ inch has been found to suffice for commonly available g-tubes/buttons. It will also be appreciated that the circumferential length of the cushion device 100 is directly related to the torso size of the user, such that different sized cushion devices can be fabricated to be worn by users of different ages, sizes and shapes. Alternatively, adjustable straps (such as straps having an adjustable length) may be included such that the cushion device 100 can be worn by users within a range of torso sizes. Preferably the width of the cushion device 100 is selected so that the device covers the torso from slightly above or at the nipple line to approximately waist level, although different widths may of course be used. Once again, cushion devices 100 having different width sizes can be fabricated for different sized users. Finally, it will be appreciated that the cushion device 100 need not be a stand-alone device, but may instead be included directly within a piece of clothing or other item of manufacture.

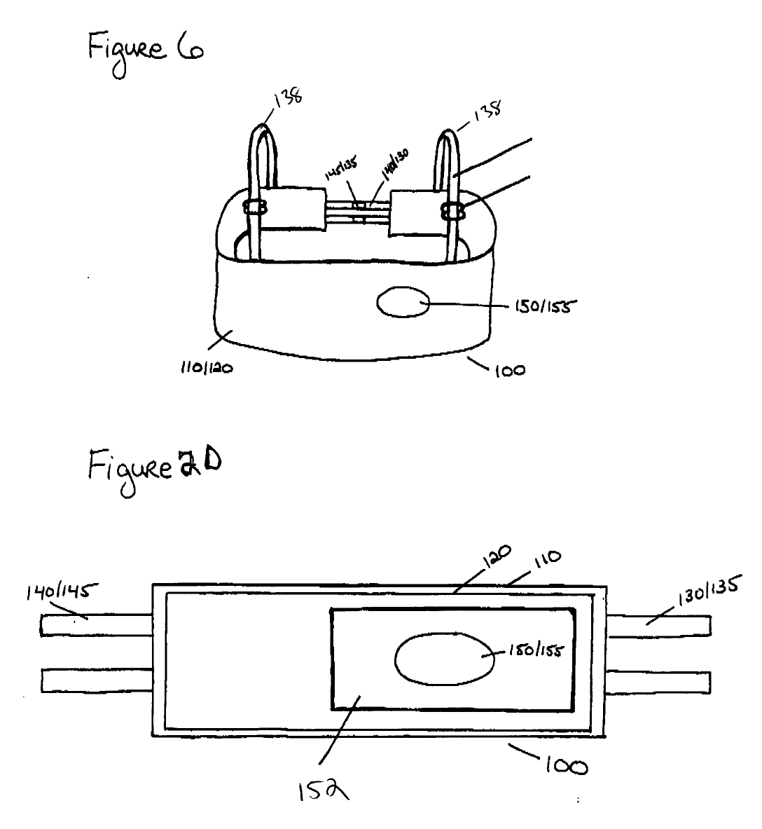

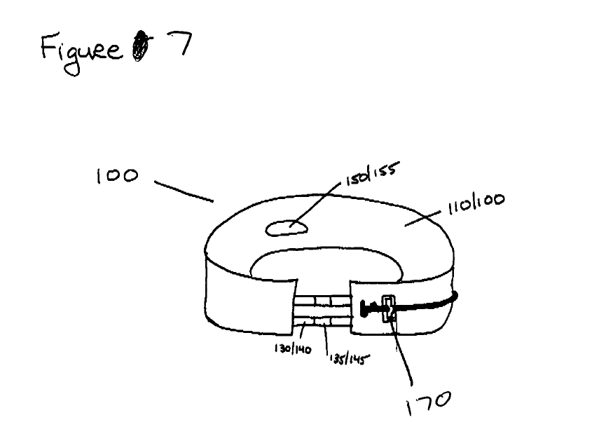

Two additional embodiments of the cushion device 100 are illustrated in FIG. 6 in FIG. 7. As shown in FIG. 6, the cushion device 100 may include two straps 138 connected to the body of the device 110. These straps 138 may be permanently or releasably attached to the body of the device 110 and are sized to fit over the wearer's shoulders. In this way, the straps 138 can be used to assist with maintaining proper positioning of the cushion device 100 on the wearer. Preferably, the straps 138 are adjustable such that the cushion device 100 can be worn by different size users. As shown in FIG. 7, the cushion device 100 may include an attachment mechanism 170, which is preferably a small piece of Velcro®, which is attached to the outside of the back of the body 110. The attachment mechanism 170 can be used to secure the end of the gastrostomy tube 20 if the tube is disconnected from the feeder but not disconnected from the button. In this way, the tube 20 can be secured to the body of the device 110 and need not hang down from the gastrostomy site 10.

While the invention has been described in connection with an exemplary embodiment thereof, it is not so limited in scope but rather is defined by the recitations in the following claims and equivalents thereof.

We claim:

1. A cushion device for a gastrostomy tube or gastrostomy button protruding from an anterior section of a user's torso, the cushion comprising:
an elongated body having opposing end sections, the end sections releasably securable to one another when the body is placed around the user, the body including a padding having a thickness at least as great as the button or the gastrostomy tube, the padding further including an open area to accommodate the button or the gastrostomy tube.

2. The cushion device of claim 1, wherein the end sections comprise one or more straps attached to the elongated body.

3. The cushion device of claim 1, wherein the end sections are releasably secured using Velcro.

4. The cushion device of claim 1, wherein the body is constructed of felt.

5. The cushion device of claim 1, wherein the padding is of a length sufficient to surround the user's anterior section.

6. The cushion device of claim 1, wherein the padding is of a length sufficient to surround the entirety of the user's torso.

7. The cushion device of claim 1, wherein the cushion device further includes a removable cover for the padding, the cover having an opening disposed over the open area.

8. The cushion device of claim 1, wherein the cushion device is included within an article of clothing worn by the user.

9. The cushion device of claim 1, wherein the cushion device includes a pair of adjustable shoulder straps.

10. The cushion device of claim 1, wherein the cushion device includes an attachment mechanism securable to the gastrostomy tube.

11. The cushion device of claim 1, wherein the elongated body includes an adhesion material which lightly adheres to a user's skin.

12. A method for encouraging the physical development of a child having a gastrostomy tube or a gastrostomy button protruding from an anterior region of the child's torso, the method comprising:
providing a cushion device around the child's torso, the cushion device including an elongated body having opposing end sections, the end sections releasably securable to one another when the body is placed around the child, the body including a padding having a thickness at least as great as the button or the gastrostomy tube, the padding further including an open area to accommodate the button or the gastrostomy tube;
securing the cushion device around the child so that the open area is disposed over the button or tube;
placing the child on the child's anterior region; and
improving mobility and muscle strengthening by allowing the child to perform an activity selected from the group consisting of crawling, rolling, playing and pivoting.

13. A method for increasing an individual's comfort with respect to a gastostomy tube or gastostomy button protruding from an anterior region of the individual's torso, comprising:
securing a cushion device around the torso, the cushion device including a padding having a thickness at least as great as the button or the gastrostomy tube, the padding further including an open area to accommodate the button or the gastrostomy tube when placed over the button or tube.
